(19) United States
(12) Patent Application Publication
(10) Pub. No.: US 2024/0092880 A1
(43) Pub. Date: Mar. 21, 2024

(54) TREATMENT OF MIGRAINE

(71) Applicant: Mark Hasleton, RAANANA (IL)

(72) Inventor: Mark Hasleton, RAANANA (IL)

(21) Appl. No.: 17/959,300

(22) Filed: Oct. 4, 2022

Related U.S. Application Data
(60) Provisional application No. 63/251,868, filed on Oct. 4, 2021.

(51) Int. Cl. C07K 16/18; A61K 31/4545; A61K 31/496; A61K 31/499; A61K 31/517; A61K 31/55; A61K 31/5513; A61K 39/395; A61P 25/06

(52) U.S. Cl. CPC C07K 16/18; A61K 31/4545; A61K 31/496; A61K 31/499; A61K 31/517; A61K 31/55; A61K 31/5513; A61K 39/3955; A61P 25/06

(57) ABSTRACT

The present invention relates to methods for the preventive treatment of migraine in a subject comprising administering to said subject that does not exhibit signs of allodynia and/or hyperalgesia at least twenty-four hours into the post-ictal phase, an anti-PACAP agent.

This application claims the benefit of U.S. Ser. No. 63/251,868, filed 4 Oct. 2021 and titled “TREATMENT OF MIGRAINE”, the contents of which are incorporated herein by reference.

### BACKGROUND

Pituitary adenylate cyclase activating polypeptide-38 (PACAP38) is a widely distributed 38 amino acid neuropeptide involved in neuroprotection, neurodevelopment, nociception and inflammation which shares a 68% homology with vasoactive intestinal peptide (VIP) and is a member of the VI P/glucagon/growth hormone releasing factor/secretin superfamily. A second 27 amino acid neuropeptide fragment of PACAP38, PACAP27, has also been identified. Both peptides share the same N-terminal 27-amino acid sequence and are produced from the same precursor protein, prepro-PACAP. Of the two, PACAP38, is the more prevalent form, representing up to 90% of PACAP forms in mammalian tissues

3 G-protein coupled receptors PACAP receptors have been described, VPAC₁, VPAC₂ and PAC₁, along with various isoforms of their receptor domains. VPAC₁ and VPAC₂ share equal affinity for both PACAP and VIP, while PAC₁ is approximately 100 fold more selective for PACAP. This has led to the suggestion that the more individualized actions of PACAP38 might be mediated through the PAC₁ receptor than through either VPAC₁ or VPAC₂.

PACAP receptors are widely distributed in the brain and peripheral organs, such as the endocrine system, gonads, sympathetic neurons, respiratory system, gastrointestinal tract, cardiovascular system, and urogenital tracts. PACAP is expressed, along with its receptors, throughout the nervous system, including the trigeminovascular system, trigeminal ganglia, trigeminal nucleus caudalis, dorsal horn of the spinal cord, brainstem, hypothalamus, pituitary, and otic and sphenopalatine ganglion.

Functionally, PACAP38 plasma levels have been identified as raised in spontaneous cluster headache and migraine attacks and normalized after successful attack abortion by sumatriptan. PACAP38 administration has also been shown to trigger migraine attacks in both preclinical studies and in migraine sufferers, a feature distinguishable from VIP. Studies have shown that PACAP38 acts by inducing trigeminal activation, probably as a direct effect rather than via a CGRP-mediated mechanism. The exact site of action of PAC₁ has not fully identified, however, it is known that administration of PACAP38, a large polypeptide of molecular weight >4000, can trigger migraine attacks without noticeably crossing the blood-brain-barrier.

The possible role of PACAP and PAC₁ in the pathogenesis of migraine and other headaches has led to the suggestion that blockade of their activities might be targets for migraine and other headache therapies. Attempts have been made to identify both PAC₁ specific antagonists or antibodies directed against the receptor along with antagonists or antibodies specifically targeted against the PACAP ligand itself. At least one such PAC₁ receptor antibody has been demonstrated preclinically to inhibit stimulus-evoked activity in the trigeminocervical complex, binding to the trigeminal ganglion and sphenopalatine ganglion but not within the central nervous system suggesting a peripheral site of action and that peripheral PAC₁ receptor inhibition might be sufficient to abort or prevent attacks of cluster headache and migraine

Therefore, methods for determining whether treatment comprising an anti-PACAP agent will be effective in the treatment of a patient who has headache or who is susceptible to headache are needed.

### SUMMARY

The present invention relates to methods for the preventive treatment of migraine in a subject comprising determining or having determined, whether the subject exhibits allodynia and/or hyperalgesia at least twenty-four hours into the post-ictal phase of the migraine, and administering to said subject that does not exhibit signs of allodynia and/or hyperalgesia at least twenty-four hours into the post-ictal phase, an anti-PACAP agent.

### DETAILED DESCRIPTION

As used herein, “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

A large body of evidence supports an important role for PACAP in the pathophysiology of migraine. This evidence gave rise to a global effort to develop a new generation of therapeutics that reduces the availability of PACAP in migraineurs. Recently, anti-PACAP agents have been found to be preclinically effective in reducing the frequency of chronic or episodic migraine.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgGI, IgG2, IgG3, IgG4, IgAI, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” or “mAb” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and, biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nat. Biotechnol, 14:309-314; Sheets et al., 1998, PNAS, (USA) 95:6157-6162; Hoogenboom and Winter, 1991, J. Mol. Biol, 227: 381; Marks et al., 1991, J. Mol. Biol., 222: 581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625, 126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol, 147 (I): 86-95; and U.S. Pat. No. 5,750,373.

As used herein, the term “calcitonin gene-related peptide” and “CGRP” refers to any form of calcitonin gene-related peptide and variants thereof that retain at least part of the activity of CGRP. For example, CGRP may be a-CGRP or β-CGRP. As used herein, CGRP includes all mammalian species of native sequence CGRP, e.g., human, canine, feline, equine, and bovine.

As used herein, an “anti-PACAP antibody” refers to an antibody that modulates PACAP biological activity, or the PACAP pathway, including downstream pathways mediated by PACAP signaling, such as receptor binding and/or elicitation of a cellular response to PACAP. The term anti-PACAP antibody encompasses both “anti-PACAP antagonist antibodies” and “anti-PACAP receptor antibodies.” In some embodiments, the anti-PACAP antibody is a monoclonal antibody (i.e., an anti-PACAP monoclonal antibody).

An “anti-PACAP antagonist antibody” refers to an antibody that is able to bind to PACAP and thereby inhibit PACAP biological activity and/or downstream pathway(s) mediated by PACAP signaling. An anti-PACAP antagonist antibody encompasses antibodies that modulate, block, antagonize, suppress or reduce PACAP biological activity, or otherwise antagonize the PACAP pathway, including downstream pathways mediated by PACAP signaling, such as receptor binding and/or elicitation of a cellular response to PACAP. In some embodiments, an anti-PACAP antagonist antibody binds PACAP and prevents PACAP binding to a PACAP receptor. In other embodiments, an anti-PACAP antagonist antibody binds PACAP and prevents activation of a PACAP receptor.

An “anti-PACAP receptor antibody” refers to an antibody that is able to bind to a PACAP receptor and thereby modulate the PACAP pathway.

As used herein, an “anti-PAC₁ antibody” refers to an antibody that modulates PAC₁ biological activity, or the PAC₁ pathway, including downstream pathways mediated by PAC₁ signaling, such as receptor binding and/or elicitation of a cellular response to PAC₁. The term anti-PAC₁ antibody encompasses both “anti-PAC₁ antagonist antibodies” and “anti-PAC₁ receptor antibodies.” In some embodiments, the anti-PAC₁ antibody is a monoclonal antibody (i.e., an anti-PAC₁ monoclonal antibody).

An “anti-PAC₁ antagonist antibody” refers to an antibody that is able to bind to PAC₁ and thereby inhibit PAC₁ biological activity and/or downstream pathway(s) mediated by PAC₁ signaling. An anti-PAC₁ antagonist antibody encompasses antibodies that modulate, block, antagonize, suppress or reduce PAC₁ biological activity, or otherwise antagonize the PAC₁ pathway, including downstream pathways mediated by PAC₁ signaling, such as receptor binding and/or elicitation of a cellular response to PAC₁. In some embodiments, an anti-PAC₁ antagonist antibody binds PAC₁ and prevents PAC₁ binding to a PAC₁ receptor. In other embodiments, an anti-PAC₁ antagonist antibody binds PAC₁ and prevents activation of a PAC₁ receptor.

An “anti-PAC₁ receptor antibody” refers to an antibody that is able to bind to a PAC₁ receptor and thereby modulate the PAC₁ pathway.

A “small molecule PACAP antagonist” refers to a small molecule which antagonises PACAP ligand binding.

A “small molecule PAC₁ antagonist” refers to a small molecule which antagonises PAC₁ ligand binding.

An “anti-PACAP agent” refers to an active agent selected from the group consisting of anti-PACAP antibodies, anti-PAC₁ antibodies, and small molecule PACAP and/or PAC₁ antagonists.

Examples of anti-PACAP agents are described in WO9114786, WO2014144632, WO2016044224, WO2016168760, WO2016168762, WO2016168768, WO2016168757, WO2017106383, WO2017106578, WO2017181031, WO2017181039, WO2018222991, WO201906574, WO2019067293, WO2019098254, WO2019140216, WO2020168068, WO2020230867, WO2020264384 and WO2021150910, each of which is which are incorporated by reference in their entirety.

As used herein, an “anti-CGRP antibody” refers to an antibody that modulates CGRP biological activity, or the CGRP pathway, including downstream pathways mediated by CGRP signaling, such as receptor binding and/or elicitation of a cellular response to CGRP. For example, an anti-CGRP antibody may block, inhibit, suppress or reduce the calcitonin gene related peptide (CGRP) pathway. The term anti-CGRP antibody encompasses both “anti-CGRP antagonist antibodies” and “anti-CGRP receptor antibodies.” In some embodiments, the anti-CGRP antibody is a monoclonal antibody (i.e., an anti-CGRP monoclonal antibody).

An “anti-CGRP antagonist antibody” refers to an antibody that is able to bind to CGRP and thereby inhibit CGRP biological activity and/or downstream pathway(s) mediated by CGRP signaling. An anti-CGRP antagonist antibody encompasses antibodies that modulate, block, antagonize, suppress or reduce CGRP biological activity, or otherwise antagonize the CGRP pathway, including downstream pathways mediated by CGRP signaling, such as receptor binding and/or elicitation of a cellular response to CGRP. In some embodiments, an anti-CGRP antagonist antibody binds CGRP and prevents CGRP binding to a CGRP receptor. In other embodiments, an anti-CGRP antagonist antibody binds CGRP and prevents activation of a CGRP receptor. Examples of anti-CGRP antagonist antibodies are provided herein.

An “anti-CGRP receptor antibody” refers to an antibody that is able to bind to a CGRP receptor and thereby modulate the CGRP pathway. Examples of anti-CGRP receptor antibodies are provided herein (e.g., erenumab).

A “gepant” refers to a small molecule CGRP antagonist. Examples of gepants are provided herein and include rimegepant, ubrogepant, vazegepant, atogepant, olcegepant, telcagepant, BI 44370 and MK-3207.

An “anti-CGRP agent” refers to an active agent selected from the group consisting of anti-CGRP antibodies and gepants.

As used herein, the terms “GI,” “antibody GI,” “TEV-48125,” and “fremanezumab” are used interchangeably to refer to an anti-CGRP antagonist antibody produced by expression vectors having deposit numbers of ATCC PTA-6867 and ATCC PTA-6866. The characterization and processes for making antibody GI (and variants thereof) are described in PCT Publication No. WO2007/054809 and WHO Drug Information 30(2): 280-1 (2016), which are hereby incorporated by reference in its entirety

The terms “ALD403,” and “eptinezumab” refer to an anti-CGRP antagonist antibody, which is a humanized IgGI monoclonal antibody from a rabbit precursor. Characterization and processes for making eptinezumab can be found in U.S. Publication No. US2012/0294797 and WHO Drug Information 30(2): 274-5 (2016), which are incorporated by reference in its entirety.

The terms “LY2951742,” and “galcanezumab” refer to an anti-CGRP antagonist antibody, which is a humanized IgG4 monoclonal antibody from a murine precursor. Characterization and processes for making galcanezumab can be found in U.S. Publication No. US2011/0305711 and WHO Drug Information 29(4): 526-7 (2015), which are incorporated by reference in its entirety. Dosing and formulations associated with galcanezumab can be found in PCT Publication No. WO 2016/205037, which is also incorporated by reference in its entirety.

The terms “AMG334,” and “erenumab” refer to an anti-CGRP receptor antibody, which is a fully humanized IgG2 antibody. Characterization and processes for making erenumab can be found in U.S. Publication No. US2010/0172895, U.S. Pat. No. 9,102,731, and WHO Drug Information 30(2): 275-6 (2016), each of which are incorporated by reference in their entireties. Dosing and formulations associated with erenumab can be found in PCT Publication No. WO 2016/171742, which is also incorporated by reference in its entirety.

The term “rimegepant” refers to a small molecule CGRP antagonist. Characterization and processes for making rimegepant can be found in U.S. Pat. Nos. 8,314,117 and 8,759,372, each of which are incorporated by reference in their entireties.

The term “ubrogepant” refers to a small molecule CGRP antagonist. Characterization and processes for making ubrogepant can be found in U.S. Pat. Nos. 8,754,096, 8,912,210 and 9,499,545, each of which are incorporated by reference in their entireties.

The term “vazegepant” refers to a small molecule CGRP antagonist. Characterization and processes for making vazegepant can be found in PCT Publication No. WO2011/123232, which is incorporated by reference in its entirety.

The term “atogepant” refers to a small molecule CGRP antagonist. Characterization and processes for making atogepant can be found in U.S. Pat. No. 8,754,096 which is incorporated by reference in its entirety.

The term “olcegepant” refers to a small molecule CGRP antagonist. Characterization and processes for making olcegepant can be found in U.S. Pat. No. 6,344,449 which is incorporated by reference in its entirety.

The term “telcagepant” refers to a small molecule CGRP antagonist. Characterization and processes for making telcagepant can be found in U.S. Pat. No. 6,953,790 which is incorporated by reference in its entirety.

The term “BI 44370” refers to a small molecule CGRP antagonist. Characterization and processes for making BI 44370 can be found in PCT Publication No. WO2005/092880 which is incorporated by reference in its entirety.

The term “MK-3207” refers to a small molecule CGRP antagonist. Characterization and processes for making MK-3207 can be found in Publication No. US2007/0265225 which is incorporated by reference in its entirety.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, intemucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylates, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(0)S(“thioate”), P(S)S (“dithioate”), (0)NR2(“amidate”), P(0)R, P(0)OR′, CO or CH2(“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. The preceding description applies to all polynucleotides referred to herein, including RNA and DNA.

Diagnosis or assessment of headache is well-established in the art. References such as the International Classification of Headache Disorders, 3rd edition (ICHD-III beta version; Cephalalgia (2013) 33(9): 629-808) can be used by a skilled practitioner to assess the type of headache experienced by a patient. Headaches within the scope of the instant invention include headaches of intracranial origin. Non-limiting examples of headaches of intracranial origin include migraine (e.g., chronic and episodic), cluster headaches and headache attributed to meningitis, an epidural bleed, a subdural bleed, a sub-arachnoid bleed, and certain brain tumors (wherein headache results from increased pressure in the skull). For example, “chronic migraine” refers to headache occurring on 15 or more days per month for more than three months, which has the features of migraine headache on at least 8 days per month, whereas “episodic migraine” refers to headache occurring less than 15 days per month, and “high frequency episodic migraine” refers to headache occurring between 8 and 14 days per month.

Skilled practitioners will be readily able to recognize a subject with any of the types of migraine headache described herein. Assessment may be performed based on subjective measures, such as patient characterization of symptoms. For example, migraine may be diagnosed based on the following criteria: 1) episodic attacks of headache lasting 4 to 72 hours; 2) with two of the following symptoms: unilateral pain, throbbing, aggravation on movement, and pain of moderate or severe intensity; and 3) one of the following symptoms: nausea or vomiting, and photophobia or phonophobia (Goadsby et al, N. Engl. J. Med. 346:257-270 2002). In some embodiments, assessment of headache (e.g., migraine) may be via headache hours, as described elsewhere herein. For example, assessment of headache (e.g., migraine) may be in terms of daily headache hours, weekly headache hours, monthly headache hours and/or yearly headache hours. In some cases, headache hours may be as reported by the subject.

The terms “patient” and “subject” are used interchangeably herein. As used herein, the patient is a human.

As used herein, “treatment” is an approach for obtaining beneficial or desired clinical results. For purposes of this invention, beneficial or desired clinical results include, but are not limited to, one or more of the following: improvement in any aspect of headache, including lessening severity, alleviation of pain intensity, and other associated symptoms, reducing frequency of recurrence, reducing frequency of headache, increasing the quality of life of those suffering from the headache, and decreasing dose of other medications required to treat the headache. Using migraine as an example, other associated symptoms include, but are not limited to, nausea, vomiting, and sensitivity to light, sound, and/or movement.

As used herein, “acute treatment” is an approach for obtaining immediate beneficial or desired clinical results. For purposes of this invention, immediate beneficial or desired clinical results include, but are not limited to, one or more of the following: an increase in pain freedom and most bothersome symptom (MBS) freedom at two hours after dosing, wherein pain freedom can be defined as a reduction of moderate or severe headache pain to no headache pain and MBS freedom as the absence of the self-identified MBS, such as photophobia, phonophobia or nausea, an increase in pain relief at 2 hours, wherein pain relied can be defined as the reduction in migraine pain from moderate or severe severity to mild or none, an increase in sustained pain freedom at 2-48 hours, a reduction in the use of rescue medication within 24 hours, and an increase in the percentage of patients reporting normal function at two hours after dosing.

As used herein, “preventive treatment” is an approach for obtaining beneficial or desired clinical results over time. For purposes of this invention, beneficial or desired clinical results over time include, but are not limited to, one or more of the following: an improvement in aspects of headache, including reducing frequency of recurrence, reducing frequency of headache, increasing the quality of life of those suffering from the headache, and decreasing dose of other medications required to treat the headache.

As used herein, “preventing” is an approach to stop headache from occurring or existing in a subject, who is susceptible to the development of headache. For example, the patient may been previously diagnosed with chronic or episodic migraine.

“Reducing headache incidence” or “reducing headache frequency” means any of reducing severity (which can include reducing need for and/or amount of (e.g., exposure to) other drugs and/or therapies generally used for this headache condition), duration, and/or frequency (including, for example, delaying or increasing time to next headache attack in an individual). As is understood by those skilled in the art, individuals may vary in terms of their response to treatment, and, as such, for example, a “method of reducing frequency of headache in an individual” reflects administering the anti-PACAP agent based on a reasonable expectation that such administration may likely cause such a reduction in headache incidence in that particular individual.

“Ameliorating” headache or one or more symptoms of headache means a lessening or improvement of one or more symptoms of headache as compared to not administering an anti-PACAP agent. “Ameliorating” also includes shortening or reduction in duration of a symptom.

As used herein, “controlling headache” refers to maintaining or reducing severity or duration of one or more symptoms of headache or frequency of headache (e.g., migraine) attacks in an individual (as compared to the level before treatment). For example, the duration or severity of head pain, or frequency of attacks is reduced by at least about any of 10%, 20%, 30%, 40%, 50%, 60%, 70%, 80%, 90% or more, in the individual as compared to the duration or severity of head pain, or frequency of attacks before treatment.

As used herein, a “headache hour” refers to an hour during which a subject experiences headache. Headache hours can be expressed in terms of whole hours (e.g., one headache hour, two headache hours, three headache hours, etc.) or in terms of whole and partial hours (e.g., 0.5 headache hours, 1.2 headache hours, 2.67 headache hours, etc.). One or more headache hours may be described with respect to a particular time interval. For example, “daily headache hours” may refer to the number of headache hours a subject experiences within a day interval (e.g., a 24-hour period). In another example, “weekly headache hours” may refer to the number of headache hours a subject experiences within a week interval (e.g., a 7-day period). As can be appreciated, a week interval may or may not correspond to a calendar week. In another example, “monthly headache hours” may refer to the number of headache hours a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache hours” may refer to the number of headache hours a subject experiences within a year interval. As can be appreciated, a year interval (e.g., a period of 365 or 366 days) may vary in terms of number of days depending upon the particular year and may or may not correspond to a calendar year.

As used herein, a “headache day” refers to a day during which a subject experiences headache. Headache days can be expressed in terms of whole days (e.g., one headache day, two headache days, three headache days, etc.) or in terms of whole and partial days (e.g., 0.5 headache days, 1.2 headache days, 2.67 headache days, etc.). One or more headache days may be described with respect to a particular time interval. For example, “weekly headache days” may refer to the number of headache days a subject experiences within a week interval (e.g., a 7-day period). As can be appreciated, a week interval may or may not correspond to a calendar week. In another example, “monthly headache days” may refer to the number of headache days a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache days” may refer to the number of headache days a subject experiences within a year interval. As can be appreciated, a year interval (e.g., a period of 365 or 366 days) may vary in terms of number of days depending upon the particular year and may or may not correspond to a calendar year.

As used herein, “delaying” the development of headache means to defer, hinder, slow, retard, stabilize, and/or postpone progression of the disease. This delay can be of varying lengths of time, depending on the history of the disease and/or individuals being treated. As is evident to one skilled in the art, a sufficient or significant delay can, in effect, encompass prevention, in that the individual does not develop headache. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of headache means initial manifestations and/or ensuing progression of the disorder. Development of headache can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of headache includes initial onset and/or recurrence.

“Responder rate” means the proportion of patients reaching at least a 50% reduction in monthly average number of migraine days during a predetermined treatment period. In one embodiment of the invention, the predetermined treatment period is 3 months. In another embodiment of the invention, the predetermined treatment period is 6 months. In yet another embodiment of the invention, the predetermined treatment period is 12 months.

Migraine can be defined by both its periodicity and its specific phases. As used herein, the “inter-ictal phase” of a migraine refers to the interval between two migraine attacks, the “pre-ictal phase” refers to the time before the headache starts, when the patient may develop premonitory symptoms, including appetite changes, thirst, yawning, or others, the “ictal phase” refers to the time period when the patient experiences headache and which last for between 4-72 hours, and the “post-ictal phase” refers to the time within the inter-ictal phase following the cessation of the headache and typically characterized by non-headache symptoms such as cognitive deficits, fatigue, and others.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, reduction in responder rate or delaying the onset of the disease, including biochemical, histological and/or behavioral symptoms of the disease, its complications and intermediate pathological phenotypes presenting during development of the disease. For therapeutic use, beneficial or desired results include clinical results such as reducing pain intensity, duration, or frequency of headache attack, reduction in responder rate and decreasing one or more symptoms resulting from headache (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “allodynia” refers to pain experienced by a patient and due to a stimulus that does not normally elicit pain (International Association for the Study of Pain, 2014-2015, “Allodynia and Hyperalgesia in Neuropathic Pain”).

As used herein, “hyperalgesia” refers to an increase in pain experienced by a patient from a stimulus that normally provokes pain (International Association for the Study of Pain, 2014-2015, “Allodynia and Hyperalgesia in Neuropathic Pain”).

Both allodynia and hyperalgesia can be determined, distinguished and quantified by one of skill in the art by methods such as, for example, quantitative sensory testing (QST) (Rolke (2006) et al. Pain 123: 231-243). Rolke et al. teaches QST reference data for obtaining the full somatosensory phenotype of a patient, in both relative and absolute terms. For example, Rolke et al. describes a test for mechanical pain sensitivity (MPS) as a means for detecting pinprick hyperalgesia. In such a test, MPS can be assessed using a set of pinprick stimuli to obtain a stimulus-response function for pinprick-evoked pain (where the strongest pinprick force is about eight-times the mean mechanical pain threshold). Subjects can be asked to give the pain a rating for each stimulus on a ‘0-100’ scale, wherein ‘0’ indicates no pain and ‘100’ indicates highest pain. A certain number of pinpricks are delivered to the subject at certain time intervals to avoid wind-up. After each pinprick, the subject provides numerical pain ratings. MPS is then calculated as the geometric mean (compound measure) of all numerical ratings for pinprick stimuli (Rolke et al. at p. 233).

As used herein, “heat allodynia and/or hyperalgesia” refers to a heat pain threshold of below 41° C., “cold allodynia and/or hyperalgesia” refers to a cold pain threshold of above 21° C. and “mechanical allodynia and/or hyperalgesia” refers to a mechanical pain threshold of below 30 g for skin indentation with calibrated von Frey hairs

As used herein, “sensitization” is the process whereby the strength of the stimulus that is needed to generate a response decrease over time, while the amplitude of the response increases.

The phrase “headache primarily experienced in a portion of the head” refers to description by the patient of having headache (experienced as, e.g., pain) in an identified part of the head. Examples of “portions of the head” include one-side periorbital, one-side temporal, one eye, a small area in the back of the head (e.g., just lateral to the midline), a small area on the top of the head, a small area in the middle of the forehead, a ‘dot’ (e.g., 10×10 mm) where the supraorbital nerve exits the skull (i.e., in the medial end of the eyebrow) and a small area across the forehead. One of skill in the art would be able to assess whether a patient is experiencing headache in a portion of the head based on the patient's description (Noseda, R. et al. (2016) Brain. 139 (7): 1971-1986).

The majority of episodic migraineurs seeking secondary or tertiary medical care exhibit signs of allodynia and/or hyperalgesia during the ictal phase of migraine, but not during the inter-ictal phase (Burstein et al. 2000b; Lipton et al. 2008; Bigal et al. 2008; Burstein et al. 2000a). In contrast, chronic migraine patients commonly exhibit sign of allodynia and/or hyperalgesia both during acute migraine attacks as well as during the inter-ictal phase. Mechanistically, allodynia is thought to be mediated by sensitization of central trigeminovascular neurons in the spinal trigeminal nucleus (Burstein et al. 1998). Furthermore, the presence of inter-ictal allodynia and/or hyperalgesia is mediated by central trigeminovascular neurons whose sensitization state does not depend on incoming pain signals from the meninges, whereas the absence of inter-ictal allodynia and/or hyperalgesia in migraine patients is explained by the existence of central trigeminovascular neurons whose sensitized state depends on pain signals that come from the periphery.

Anti-PACAP agents are unlikely to cross the blood-brain-barrier and inhibit the central trigeminovascular neurons directly. It has now been determined that the therapeutic ability of anti-PACAP agents dictate that in some episodic, most likely high frequency, and chronic migraine patients, central sensitization and allodynia and/or hyperalgesia remain dependent of pain signals that originate in the meninges and that patients who will respond to these agents will be those in which the ongoing peripheral input is required to maintain the central sensitization, whereas the non-responders will be those in which the ongoing peripheral input is not required to maintain the central sensitization. Therefore, the peripheral site of action of anti-PACAP agents will allow these medications to provide preventive treatment for both episodic and chronic migraine patients whose state of central sensitization depends on pain signals that arrive from the meninges but not in those patients in which the state of central sensitization is independent of the pain signals that arrive from the meninges. Surprisingly, it has now been determined that this determination of dependence, or independence on pain signals arriving from the meninges, i.e. the absence or presence of inter-ictal allodynia and/or hyperalgesia, can be made at least twenty-four hours into the post-ictal phase of the migraine.

Provided herein are methods of treating migraine in a subject comprising determining or having determined whether the subject exhibits allodynia and/or hyperalgesia at least twenty-four hours into the post-ictal phase of the migraine, and administering an anti-PACAP agent to the subject that does not exhibit signs of allodynia and/or hyperalgesia at least twenty-four hours into the post-ictal phase of the migraine. In one embodiment of the invention, the treatment is preventive. In another embodiment of the invention, the treatment is acute.

Also provided herein are methods of treating headache in a subject comprising determining or having determined whether the subject exhibits allodynia and/or hyperalgesia at least twenty-four hours into the post-ictal phase of the headache, and administering an anti-PACAP agent to the subject that does not exhibit signs of allodynia and/or hyperalgesia at least twenty-four hours into the post-ictal phase of the headache. In one embodiment of the invention, the treatment is preventive. In another embodiment of the invention, the treatment is acute.

Also provided herein are methods of treating cluster headache in a subject comprising determining or having determined whether the subject exhibits allodynia and/or hyperalgesia at least twenty-four hours into the post-ictal phase of the headache, and administering an anti-PACAP agent to the subject that does not exhibit signs of allodynia and/or hyperalgesia at least twenty-four hours into the post-ictal phase of the headache. In one embodiment of the invention, the treatment is preventive. In another embodiment of the invention, the treatment is acute.

In one embodiment of the invention, the subject has been determined whether they exhibit allodynia and/or hyperalgesia at least twenty-four hours into the post-ictal phase of their migraine by a suitably qualified healthcare professional. In one embodiment of the invention, the determination that the subject exhibits allodynia and/or hyperalgesia is performed by a suitably qualified healthcare professional at least twenty-four hours into the post-ictal phase of the subject's migraine.

In one embodiment of the invention, the determination of whether the subject exhibits allodynia and/or hyperalgesia is at least twenty-four hours into the post-ictal phase of the migraine. In another embodiment of the invention, the determination of whether the subject exhibits allodynia and/or hyperalgesia is at least thirty-six hours into the post-ictal phase of the migraine. In another embodiment of the invention, the determination of whether the subject exhibits allodynia and/or hyperalgesia is at least forty-eight hours into the post-ictal phase of the migraine. In another embodiment of the invention, the determination of whether the subject exhibits allodynia and/or hyperalgesia is at least sixty hours into the post-ictal phase of the migraine. In another embodiment of the invention, the determination of whether the subject exhibits allodynia and/or hyperalgesia is at least seventy-two hours into the post-ictal phase of the migraine.

In one embodiment of the invention, the subject is determined to not exhibit allodynia and/or hyperalgesia at least twenty-four hours into the post-ictal phase of the migraine.

In one embodiment of the invention, the subject determined to not exhibit allodynia and/or hyperalgesia at least twenty-four hours into the post-ictal phase of the migraine is treated by the administration of an anti-PACAP agent. In one embodiment of the invention, this treatment is preventive. In another embodiment of the invention, this treatment is acute.

In one embodiment of the invention, the subject suffers from episodic migraine. In one embodiment of the invention, the subject suffers from high frequency episodic migraine. In another embodiment of the invention, the subject suffers from chronic migraine.

In one embodiment of the invention, the subject is administered an anti-PACAP agent as the sole medication. In another embodiment of the invention, the subject is administered an anti-PACAP agent in combination with an anti-CGRP agent. In one embodiment of the invention, the combination of an anti-PACAP agent and an anti-CGRP agent is administered in a single pharmaceutical dosage form. In another embodiment of the invention, the combination of an anti-PACAP agent and an anti-CGRP agent is administered in separate pharmaceutical dosage forms.

In one embodiment of the invention, the determination of whether the subject exhibits allodynia and/or hyperalgesia is at least twenty-four hours into the post-ictal phase of the migraine is by quantitative sensory testing (QST). In another embodiment of the invention, the determination of whether the subject exhibits allodynia and/or hyperalgesia is at least twenty-four hours into the post-ictal phase of the migraine is by questionnaire. In another embodiment of the invention, the determination of whether the subject exhibits allodynia and/or hyperalgesia is at least twenty-four hours into the post-ictal phase of the migraine is by both quantitative sensory testing (QST) and questionnaire. In one embodiment of the invention, the QST and/or questionnaire are determined at a healthcare facility. In another embodiment of the invention, the QST and/or questionnaire are determined at the subject's place of residence.

In one embodiment of the invention, the determination of whether the subject exhibits allodynia and/or hyperalgesia is a determination of whether the subject exhibits cephalic allodynia and/or hyperalgesia. In another embodiment of the invention, the determination of whether the subject exhibits allodynia and/or hyperalgesia is a determination of whether the subject exhibits cephalic and extracephalic allodynia and/or hyperalgesia. In one embodiment of the invention, the determination of whether the subject exhibits allodynia and/or hyperalgesia is the determination of whether the subject exhibits cephalic but not extracephalic allodynia and/or hyperalgesia. As used herein, the term “cephalic allodynia and/or hyperalgesia” refers to allodynia and/or hyperalgesia at sites on the head, such as, for example, the periorbital and temporal regions, and the term “extracephalic allodynia and/or hyperalgesia” refers to allodynia and/or hyperalgesia at sites other than the head, such as, for example, the forearm or lower leg.

Quantitative Sensory testing (QST) should be performed preferably in a quiet room away from noise and distraction. There, patients should be allowed to choose their most comfortable position (sitting on a chair or lying in bed) during the sensory testing. In each testing session, pain thresholds to hot and mechanical stimulation are determined in the skin over the site to where the pain is referred to, with the periorbital and temporal regions being the most common sites tested. Heat skin stimuli should be delivered through a 30×30 mm² thermode (Q-Sense 2016, Medoc) attached to the skin at a constant pressure and the participant's pain thresholds determined by using the Method of Limit analysis.

Allodynia testing should be performed to determine pain thresholds with the skin allowed to adapt to a temperature of 32° C. for 5 minutes and then warmed up at a slow rate (1° C./sec) until pain sensation is perceived, at which moment the subject will be allowed to stop the stimulus by pressing a button on a patient response unit. Heat stimuli should be repeated three times each and the mean of recorded temperatures will be considered threshold. Pain threshold to mechanical stimuli can be determined by using a set of up to 20 calibrated von Frey hairs (VFH, Stoelting). Each VFH monofilament is assigned a scalar number in an ascending order and each monofilament should be applied to the skin 3 times (for 2 sec). The smallest VFH number capable of inducing pain at two out of three trials will be considered as threshold. Skin sensitivity can also be determined by recording the subject's perception of soft skin brushing, which is a dynamic mechanical stimulus, as distinguished from the VFH, which is a static mechanical stimulus.

Hyperalgesia testing should be performed to determine when a painful stimulus is perceived as more painful than usual. 3 supra-threshold heat and mechanical stimuli should be applied to the skin. The value of the supra-threshold stimulus can be determined during the allodynia testing which the subject will have already undertaken. In this test, the skin should be exposed to 3 supra-threshold stimuli (1-above-threshold), each lasting 10 seconds and separated by 10 seconds (i.e., inter-stimulus interval of 10 seconds). At the end of each stimulus, the patient should have 10 seconds to identify the intensity of the pain using a visual analog scale (VAS) of 0-10 (o=no pain, 10=most imaginable pain). A similar test can be administered using supra-threshold mechanical stimulation.

In one embodiment of the invention, the determination of whether the subject exhibits allodynia and/or hyperalgesia is by a determination of whether the subject has a heat pain threshold of below 41° C. and/or a cold pain threshold of above 21° C. and/or a mechanical pain threshold of below 30 g for skin indentation with calibrated von Frey hairs.

In one embodiment of the invention, the subject is determined to exhibit allodynia and/or hyperalgesia by exhibiting a heat pain threshold of below 41° C. and/or a cold pain threshold of above 21° C. and/or a mechanical pain threshold of below 30 g for skin indentation with calibrated von Frey hairs.

In one embodiment of the invention, the subject is determined to not exhibit allodynia and/or hyperalgesia by exhibiting a heat pain threshold of above 40° C. and/or a cold pain threshold of above 20° C. and/or a mechanical pain threshold of above 30 g for skin indentation with calibrated von Frey hairs.

In one embodiment of the invention, the questionnaire is specifically designed to capture the presence or absence of inter-ictal allodynia and/or hyperalgesia. In one embodiment of the invention, the questionnaire is incorporated as part of an e-diary. In one embodiment of the invention, the e-diary is recorded daily by the subject over a time period of at least seven days beginning at least twenty-four hours into the post-ictal phase of the migraine.

A specifically designed questionnaire could be a variation of the Allodynia Symptom Checklist (ASC-12) (Lipton R B et al 2008) which has been modified for the inter-ictal phase of a migraine rather than for the ictal phase where the ASC-12 is typically used. Such modifications might result in the removal of questions relating to wearing necklaces or contact lenses and the scaling might be ranked similarly to the ASC-12 or it might be a more simplified ranking of never/rarely (score=0) and at least some of the time (score=0). With such modifications, a finding of no allodynia might relate to a score of 0, of 1, of 2, of 3, of 4 or of 5.

In one embodiment of the invention, the determination of the absence of allodynia and/or hyperalgesia by the review of the questionnaire by a suitably qualified healthcare professional. In one embodiment of the invention, the determination of the absence of allodynia and/or hyperalgesia is by a questionnaire score of no more than 5. In one embodiment of the invention, the determination of the absence of allodynia and/or hyperalgesia is by a questionnaire score of no more than 5, no more than 4, no more than 3, no more than 2, no more than 1 or a questionnaire score of 0.

In one embodiment of the invention, the subject was determined at least twenty-four hours into the post-ictal phase of the migraine to exhibit heat allodynia and/or hyperalgesia in the absence of both cold and mechanical allodynia and/or hyperalgesia. In another embodiment of the invention, the subject was determined at least twenty-four hours into the post-ictal phase of the migraine to exhibit both heat and cold allodynia and/or hyperalgesia and in the absence of mechanical allodynia and/or hyperalgesia. In another embodiment of the invention, the subject was determined at least twenty-four hours into the post-ictal phase of the migraine to exhibit both heat and mechanical allodynia and/or hyperalgesia and in the absence of cold allodynia and/or hyperalgesia. In another embodiment of the invention, the subject was determined at least twenty-four hours into the post-ictal phase of the migraine to exhibit cold allodynia and/or hyperalgesia in the absence of both heat and mechanical allodynia and/or hyperalgesia. In another embodiment of the invention, the subject was determined at least twenty-four hours into the post-ictal phase of the migraine to exhibit both cold and mechanical allodynia and/or hyperalgesia and in the absence of heat allodynia and/or hyperalgesia. In yet another embodiment of the invention, the subject was determined at least twenty-four hours into the post-ictal phase of the migraine to exhibit mechanical allodynia and/or hyperalgesia in the absence of both heat and cold allodynia and/or hyperalgesia.

In one embodiment of the invention, the subject is determined to exhibit allodynia and/or hyperalgesia at least twenty-four hours into the post-ictal phase of the migraine. In such an embodiment, a second determination of the subject's allodynia and/or hyperalgesia can be performed when the subject has been migraine free for at least twenty-four hours. In one embodiment of the invention, the second determination of the subject's allodynia and/or hyperalgesia occurs when the subject has been migraine free for at least twenty-four, at least forty-eight, at least seventy-two, at least eighty-four, at least ninety-six, at least one hundred and eight or at least one hundred and twenty hours.

Provided herein are methods of treating migraine in a subject comprising determining or having determined that a subject exhibits allodynia and/or hyperalgesia at least twenty-four hours into the post-ictal phase of the migraine, then determining or having determined that a subject exhibits allodynia and/or hyperalgesia when migraine free for at least twenty-four hours and administering an anti-PACAP agent to the subject that does not exhibit signs of allodynia and/or hyperalgesia when migraine free for at least twenty-four hours and wherein the determination of allodynia and/or hyperalgesia when migraine free for at least twenty-four hours is by questionnaire. In one embodiment of the invention, the treatment is preventive. In another embodiment of the invention, the treatment is acute.

Provided herein are methods of treating migraine in a subject comprising determining or having determined that a subject exhibits allodynia and/or hyperalgesia at least twenty-four hours into the post-ictal phase of the migraine, then determining or having determined that a subject exhibits allodynia and/or hyperalgesia when migraine free for at least forty-eight hours and administering an anti-PACAP agent to the subject that does not exhibit signs of allodynia and/or hyperalgesia when migraine free for at least forty-eight hours and wherein the determination of allodynia and/or hyperalgesia when migraine free for at least forty-eight hours is by questionnaire. In one embodiment of the invention, the treatment is preventive. In another embodiment of the invention, the treatment is acute.

Provided herein are methods of treating migraine in a subject comprising determining or having determined that a subject exhibits allodynia and/or hyperalgesia at least twenty-four hours into the post-ictal phase of the migraine, then determining or having determined that a subject exhibits allodynia and/or hyperalgesia when migraine free for at least seventy-two hours and administering an anti-PACAP agent to the subject that does not exhibit signs of allodynia and/or hyperalgesia when migraine free for at least seventy-two hours and wherein the determination of allodynia and/or hyperalgesia when migraine free for at least seventy-two hours is by questionnaire. In one embodiment of the invention, the treatment is preventive. In another embodiment of the invention, the treatment is acute.

In one embodiment of the invention, the preventative treatment comprises a reduction in responder rate, i.e., a reduction in the proportion of patients reaching at least a 50% reduction in monthly average number of migraine days during the treatment period. In another embodiment of the invention, the preventive treatment comprises a reduction in the number of monthly migraine headache days over a treatment period of at least three months. In another embodiment of the invention, the preventive treatment comprises a reduction in the use of acute headache medication. In another embodiment of the invention, the preventive treatment comprises an improvement in the subject's functionality. In another embodiment of the invention, wherein the preventive treatment comprises an improvement in the subject's Quality of Life (QoL). In another embodiment of the invention, the preventive treatment comprises an improvement in the subject's headache severity. In another embodiment of the invention, the preventive treatment comprises a reduction in the number of monthly non-migraine headache days over a treatment period of at least three months. In yet another embodiment of the invention, the preventive treatment comprises a reduction in the subject's photophobia, phonophobia and/or nausea.

In one embodiment of the invention, the acute treatment comprises an increase in pain freedom. In another embodiment of the invention, the acute treatment comprises an increase in most bothersome symptom freedom. In another embodiment of the invention, the acute treatment comprises an increase in pain freedom and most bothersome symptom freedom. In another embodiment of the invention, the acute treatment comprises an increase in pain relief. In another embodiment of the invention, the acute treatment comprises an increase in sustained pain freedom. In another embodiment of the invention, the acute treatment comprises a decrease in use of rescue medication. In yet another embodiment of the invention, the acute treatment comprises an increase in normal functioning.

In one embodiment of the invention, the subject administered an anti-PACAP agent remains free of allodynia and/or hyperalgesia from at least twenty-four hours into the post-ictal phase of the migraine and for at least three months after initiation of the treatment. In one embodiment of the invention, the subject administered an anti-PACAP agent remains free of allodynia and/or hyperalgesia from at least twenty-four hours into the post-ictal phase of the migraine and for at least three months, at least four months, at least five months, at least six months, at least seven months, at least eight months, at least nine months, at least ten months, at least eleven months, or at least twelve months after initiation of the treatment.

In one embodiment of the invention, the anti-PACAP agent is administered while the subject is migraine free.

This invention will be better understood by reference to the Examples, which follow. Those of skill in the art will readily appreciate that the specific experiments detailed are only illustrative of the invention as described more fully in the claims which follow thereafter.

### Examples

### Example 1: A Clinical Study of Anti-PACAP Agent Responder Rate

Thirty high frequency episodic migraine, anti-PACAP agent naïve, patients undergo QST testing at least twenty-four hours into the post-ictal phase of their migraine and complete a thirty-day e-diary questionnaire before initiation of therapy with an anti-PACAP agent. After three months of treatment with the anti-PACAP agent, the patients are reviewed to determine whether they had responded to the agent. Effective treatment is determined by a number of factors including reductions in headache intensity and the frequency of headaches, throbbing, photophobia, phonophobia and nausea. Responders are defined as those patients reaching at least a 50% reduction in monthly average number of migraine days during the 3-month treatment period. Following determination of response to the treatment, the findings are compared together with the previously blinded allodynia/hyperalgesia assessments. For those patients who demonstrated allodynia and/or hyperalgesia in QST testing at least twenty-four hours into the post-ictal phase of the migraine, completion of the allodynia and/or hyperalgesia determination is made based on e-diary questionnaire answers when the patients were migraine free for at least seventy-two hours.

1-37. (canceled)

38. A method for the preventative treatment of migraine in a human subject in need thereof, comprising
a. determining or having determined whether the subject exhibits allodynia and/or hyperalgesia at least twenty-four hours into the post-ictal phase of the migraine, and
b. administering to said subject that does not exhibit signs of allodynia and/or hyperalgesia at least twenty-four hours into the post-ictal phase, an anti-PACAP agent.

39. The method of claim 38, wherein the subject suffers from episodic migraine.

40. The method of either of claim 39, wherein the subject suffers from high frequency episodic migraine.

41. The method of claim 38, wherein the subject suffers from chronic migraine.

42. The method of claim 38, wherein the determination is at least forty-eight hours into the post-ictal phase of the migraine.

43. The method of claim 38, wherein the subject was determined at least twenty-four hours into the post-ictal phase of the migraine to have a heat pain threshold of below 41° C. and/or a cold pain threshold of above 20° C. and/or a mechanical pain threshold of below 30 g for skin indentation with calibrated von Frey hairs.

44. The method of claim 38, wherein the subject was determined at least twenty-four hours into the post-ictal phase of the migraine to exhibit heat allodynia and/or hyperalgesia in the absence of both cold and mechanical allodynia and/or hyperalgesia.

45. The method of claim 38, wherein the determination that the subject does not exhibit signs of allodynia and/or hyperalgesia at least twenty-four hours into the post-ictal phase of the migraine is determined by quantitative sensory testing (QST).

46. The method of claim 45, wherein the QST is determined at a healthcare facility.

47. The method of claim 45, wherein the QST is determined at the subject's place of residence.

48. The method of claim 38, wherein the determination of allodynia and/or hyperalgesia is by review of a questionnaire completed by the subject.

49. The method of claim 48, wherein the questionnaire is specifically designed to capture the presence or absence of interictal allodynia and/or hyperalgesia.

50. The method of claim 48, wherein the questionnaire is part of an e-diary.

51. The method of claim 50, wherein the e-diary is recorded daily by the subject over a time period of at least seven days beginning at least twenty-four hours into the post-ictal phase of the migraine.

52. The method of claim 38, wherein the determination of the absence of allodynia and/or hyperalgesia is by a questionnaire score of no more than 5.

53. The method of claim 38, further comprising the administration of an anti-CGRP agent selected from the group consisting of rimegepant, ubrogepant, vazegepant, atogepant, olcegepant, telcagepant, B144370, MK-3207, fremanezumab, erenumab, eptinezumab and galcanezumab.

54. The method of claim 38, wherein the subject was determined at least twenty-four hours into the post-ictal phase of the migraine to exhibit cephalic allodynia and/or hyperalgesia but not to exhibit extracephalic allodynia and/or hyperalgesia.

55. The method of claim 38, wherein the subject administered an anti-PACAP agent remains free of allodynia and/or hyperalgesia from at least twenty-four hours into the post-ictal phase of the migraine and for at least three months after initiation of the treatment.

56. The method of claim 38, wherein the anti-PACAP agent is administered while the subject is migraine free.

57. A method for the preventative treatment of migraine in a human subject in need thereof, comprising
a. determining or having determined whether the subject exhibits allodynia and/or hyperalgesia at least twenty-four hours into the post-ictal phase of the migraine;
b. determining or having determined whether the subject, that does exhibit signs of allodynia and/or hyperalgesia at least twenty-four hours into the post-ictal phase of the migraine, exhibits allodynia and/or hyperalgesia when migraine free for at least seventy-two hours;
c. administering to said subject that does not exhibit signs of allodynia and/or hyperalgesia when migraine free for at least seventy-two hours, an anti-PACAP agent; and
wherein the determination of allodynia and/or hyperalgesia when migraine free for at least seventy-two hours is by questionnaire.
